(19) United States
(12) Patent Application Publication
(10) Pub. No.: US 2019/0320628 A1
(43) Pub. Date: Oct. 24, 2019

(54) A NON-HUMAN ANIMAL MODEL OF NEURODEGENERATIVE DISORDERS

(71) Applicant: Expesicor LLC, Kalispell, MT (US)

(72) Inventor: Braxton A. Norwood, Kalispell, MT (US)

(21) Appl. No.: 16/474,647

(22) PCT Filed: Dec. 28, 2017
(86) PCT No.: PCT/US2017/068657

§ 371 Date: Jun. 28, 2019

Related U.S. Application Data
(60) Provisional application No. 62/439,480, filed on Dec. 28, 2016.

(51) Int. Cl. A01K 67/027; G01N 33/50

(52) U.S. Cl. CPC A01K 67/027; G01N 33/5058; A01K 2207/20; A01K 2267/02; A01K 2267/0318; A01K 2227/105

(57) ABSTRACT

Non-human animal models of neurodegenerative disorders for use in determining efficacy of anti-neurodegenerative, anti-epileptogenic and disorder-modifying pharmaceutical compositions and/or therapies.

### CROSS-REFERENCE TO RELATED APPLICATIONS

This application depends from and claims priority to U.S. Provisional Application No. 62/439,480 filed Dec. 28, 2016, the entire contents of which are incorporated herein by reference.

### TECHNICAL FIELD

The presently-disclosed subject matter generally relates to non-human animal models of neurodegenerative disorders. In particular, the presently disclosed subject matter relates to nonhuman animal models of epilepsy and related seizure disorders. More particularly, the presently disclosed subject matter relates to non-human animal models of epilepsy for use in determining efficacy of anti-neurodegenerative, anti-epileptogenic and disorder-modifying pharmaceutical compositions.

### BACKGROUND

Epilepsy is a common neurological disorder characterized by recurrent, unprovoked seizures. It affects about 1% of the world population, with around 2.4 million new patients diagnosed in one year (see for example, Bernard S. Chang et al. N Engl J Med 2003; 349:1257-66). Temporal lobe epilepsy (hereinafter designated as “TLE”) is the most common form of epilepsy in humans, in which seizures originate in the temporal lobe and is generally assumed to be caused by a brain insult. TLE is characterized by pronounced hippocampal atrophy and limited extrahippocampal damage as well as seizures that originate in the hippocampus and/or closely related structures after a prolonged seizure-free or “latent period”, which is the time between an epilepsy-inducing event and the first spontaneous seizure (See, for example, Susan S. Spencer et al. Epilepsia, 35(4):721-727, 1994. Raven Press, Ltd., New York).

Understanding the mechanisms underlying neurodegenerative disorders, epileptogenesis, seizures in epilepsy and related disorders, as well as further developing anti-neurodegenerative and anti-epileptic pharmaceutical compositions, cannot be fully acquired in clinical studies with humans. Thus, using non-human animal models are required. However, the non-human animal models need to closely and reliably mimic human neurodegenerative disorder or epilepsy and to predict the human response to drugs. Thus, non-human animal models need to display similar clinical conditions as humans and replicate the characteristics of human neurodegenerative or epilepsy such as hippocampal sclerosis and spontaneous hippocampal-onset seizures after a latent period.

Recurrent seizures have been induced in non-humans, in particular rodents, by using chemoconvulsants such as kainic acid (hereinafter designated as “KA”). KA is a glutamate analog that induces acute seizures and neurodegeneration. KA is used to model human TLE in animals, most commonly in rodents (see e.g., Levesque M. & Avoli M., Neurosci. Biobehay. Rev. 2013, 37, 2887-2899). KA exerts its effects through activation of kainate receptors, which are a type of ionotropic glutamate receptor, and also through activation of AMPA receptors, for which it is a partial agonist (Watkins J. C. & Evans R. H., Annu. Rev. Pharmacol. Toxicol. 1981, 21, 165-204).

The original KA model of epilepsy was developed by Ben-Ari and colleagues (Ben-Ari Y. & Lagowska J., C. R. Acad. Sci. Hebd. Seances Acad. Sci. D 1978, 287, 813-816; Ben-Ari Y. et al., Brain Res. 1979, 163, 176-179). In this model, behavioral seizures and neurodegeneration in the dorsal hippocampus were induced by intra-amygdaloid injections of KA. Since then, several KA-based animal models of epilepsy have been developed with the goal of inducing a period of severe, prolonged seizures, i.e. status epilepticus (hereinafter designated as “SE”), particularly convulsive status epilepticus (hereinafter designated as “cSE”). SE, whether convulsive or not, is typically fatal without pharmacologic intervention (Levesque M. et al., J. Neurosci. Methods 2015, 260, 45-52). Subsequently developed model systems were aimed at reducing variability between the animals and reducing mortality without preventing cSE and later epilepsy (Hellier J. L. & Dudek F. E., Curr. Protoc. Neurosci. 2005; PubMed listing).

For example, in one model KA is applied intracerebrally, i.e. into the hippocampus (see, for example, Arabadzisz D. et al., Exp. Neurol. 2005, 194, 76-90). In this study, mice were stereotaxically injected under general anesthesia (Equitesin, 4 ml/kg i.p.) with either 50 nl of a 20 mM solution of KA in 0.9% NaCl or the same amount of NaCl solution (control mice) into the right CA1 area of the dorsal hippocampus. This approach induces severe hippocampal sclerosis and spontaneous seizures. However, this animal model has several disadvantages: it causes cSE that requires pharmacologic termination, hippocampal injury is variable and extensive damage to the extrahippocampal regions occurs, there is a high non-responder rate and a highly variable seizure rate, the model is elaborate and its implementation is costly, and its results tend to differ between research experiments.

Ben-Ari and colleagues studied yet another animal model based on systemic KA injection, i.e. a single intraperitoneal or subcutaneous injection (Ben-Ari Y. et al., Neuroscience 1980, 5, 515-528). In this study, Wistar rats were anaesthetized with equithesin (3 ml/kg) and placed in a stereotaxic frame, and KA (0.4-2 μg dissolved in 0.1 to 0.4 μl phosphate buffer solution, pH 7.4) was unilaterally injected, under sterotaxic guidance, into the right amygdala. The animals suffered from SE that needed to be terminated by administration of a large dose of diazepam (20 mg/kg). When SE is successfully induced, extensive extrahippocampal neuron loss occurs accompanied by extensive bilateral gliosis, brain edema and neuron loss in the piriform and entorhinal cortices, olfactory bulb, substantia nigra, thalamus, and mesencephalon. This animal model does not provide any control over the KA bioavailability in the brain and there is high variability in the neuropathology and up to 30% mortality while still having 20-40% non-responders among the surviving animals.

Hellier J. L. & Dudek F. E. (Curr. Protoc. Neurosci. 2005) described another animal model based on multiple low-dose intraperitoneal injections. The protocol uses an initial KA dose of 5 mg/kg body weight, whereas subsequent doses of KA are tailored to ensure that each rat survives the treatment protocol and develops spontaneous seizures. Rats may respond differently to the initial 5 mg/kg KA injection, with some animal having seizures or wet dog shakes whereas others have no apparent motor responses. According to this study, 1 h after the initial KA injection, the severity of seizure-like activity and the behavior of each rat are used to determine the subsequent amount of KA injection, requiring an assessment every 30 or 60 min to ensure that each rat receives the correct amount of kainite in order to experience more than three consecutive hours of cSE and to survive the treatment protocol. In this model there is no control over KA bioavailability in the brain; when doses are administered over several hours the amount of KA has to be adjusted to each animal which necessitates monitoring of the individual animals; and the mortality rate is around 15%.

Williams and colleagues characterized a “repeated low-dose KA model” (Williams P. A. et al., J. Neurosci. 2009, 29, 2103-2112). In this protocol, intrahippocampal electrodes were implanted into male Sprague Dawley rats. One to two weeks after the implantation surgery, rats were injected with KA (5 mg kg⁻¹ h⁻¹) diluted in sterile 0.9% NaCl solution at 2.5 mg/ml. The rats were continuously monitored for electrographic and convulsive motor seizures. Hourly KA treatment continued in animals with convulsive seizures until more than 10 convulsive seizures per hour were evoked. This SE was maintained for at least 3 h. This model requires constant monitoring of the test animals to ensure that the animals are not overdosed and pharmacologic termination of SE, which is otherwise fatal.

Although the use of KA to model human epilepsy has proven valuable, substantial drawbacks persist, including inducement of SE or cSE that is inherently capricious and uncontrollable, high mortality (up to 50%), variable neuropathology, erratic latency to spontaneous epilepsy (first seizures can occur weeks apart in animals that received identical treatment), and non-responders (up to 50% of surviving animals never exhibit spontaneous seizures). Furthermore, existing animal models for epilepsy do not reliably recapitulate essential characteristics of the human condition, namely the relevant neuropathology and presence of a latent period before spontaneous hippocampal-onset seizures. Additionally, the relevance of CSE to the human condition is dubious, as most humans with epilepsy never experience it.

Consequently, there is a continuing need in the art for a simple and reliable, non-human animal model for neurodegenerative disorders, in particular epilepsy and/or TLE, that does not involve inducing SE and/or cSE in the non-human animal and thus prevents or reduces its inherent complications. Further, there is a need for a non-human animal model wherein the non-human animals have no significant morbidity, mortality, or a large proportion of non-responders. There is also a need for a non-human animal model wherein SE and/or cSE are blocked without stopping seizure activity. In particular, there is a need to create a non-human animal model in which the effects of KA can be targeted to the hippocampus wherein hippocampal seizures, including acute, focal hippocampal seizures, last for several hours and are self-terminating. Additionally, there is a need for a non-human animal model that reliably mimics the human neurodegenerative conditions, in particular, the defining characteristics of epilepsy and/or acquired mesial TLE, such as hippocampal sclerosis and spontaneous hippocampal-onset seizures after a prolonged seizure-free period.

There is also a need for a method for creating a non-human animal model for neurodegenerative disorders, in particular epilepsy and/or TLE, which relies on a single administration of KA, which does not require intensive monitoring and care of the animals and where there is no significant variability in the response to KA.

Furthermore, there is a need for a method of assaying the anti-neurodegenerative and/or the anti-epileptogenic efficacy of a compound, a pharmaceutical composition, or therapies by using such a reliable non-human animal model.

Additionally, there is a need for a pharmaceutical combination and/or composition for use in inducing neurodegenerative disorder, in particular, epileptic seizures in a non-human animal that does not involve inducing SE and/or cSE in the non-human animal and thus prevents or reduces its inherent complications as previously described.

Accordingly, the present invention provides such non-human animal models, pharmaceutical combinations, and methods that solve one or more of the problems mentioned above. Other features and advantages of the invention will be apparent from the following description and from the claims.

### SUMMARY

It is understood that both the following summary and the detailed description are exemplary and explanatory and are intended to provide further explanation of the disclosure as claimed. Neither the summary nor the description that follows is intended to define or limit the scope of the disclosure to the particular features mentioned in the summary or description.

Some embodiments of the present disclosure provide a non-human animal which has been administered a pharmaceutical combination comprising 10.0-30.3 mg of kainic acid (KA) per kg of the non-human animal and 0.25-1.4 mg of lorazepam per kg of the non-human animal, wherein the non-human animal exhibits a neurodegenerative disorder by said administration.

In some aspects of the previous embodiments of a non-human animal, the neurodegenerative disorder comprises epilepsy, brain injury, spinal cord injury, bipolar disorder, trigeminal neuralgia, attention-deficit hyperactivity disorder, partial seizures, adjunctive therapy for partial, myoclonic, tonic-clonic seizures, schizophrenia, neuropathic pain, seizures, Tourette syndrome, Alzheimer's disease, autism, anxiety disorder, mania, phantom limb syndrome, complex regional pain syndrome, paroxysmal extreme pain disorder, neuromyotonia, intermittent explosive disorder, borderline personality disorder, myotonia congenita, Frontotemporal dementia, multiple sclerosis, Amyotrophic Lateral Sclerosis, Parkinson's disease, and Huntington's disease, traumatic brain injury, and post-traumatic stress disorder. In certain aspects of the previous embodiments of a non-human animal, the neurodegenerative disorder comprises epilepsy. In some aspects of the previous embodiments of a non-human animal, the non-human animal does not exhibit status epilepticus and/or convulsive status epilepticus. In further aspects of the previous embodiments of a non-human animal, the non-human animal exhibits hippocampal sclerosis and spontaneous hippocampal-onset seizures.

In other aspects of the previous embodiments of a non-human animal, the administered pharmaceutical combination comprises a first composition comprising 10.0-30.3 mg of KA per kg of the non-human animal and a second composition comprising 0.25-1.4 mg of lorazepam per kg of the non-human animal. In further aspects of the previous embodiments of a non-human animal, the first composition comprising 10.0-30.3 mg of KA per kg of the non-human is administered subcutaneously in a single dose, and the second composition comprising 0.25-1.4 mg of lorazepam per kg of the non-human animal is administered subcutaneously in a single dose. In certain aspects of the previous embodiments of a non-human animal, the first composition comprising 10.0-30.3 mg of KA per kg of the non-human and the second composition comprising 0.25-1.4 mg of lorazepam per kg of the non-human are administered together in a single dosage form. In even further aspects of the previous embodiments of a non-human animal, the non-human animal is a rat.

Some embodiments of the present disclosure provide a pharmaceutical combination for use in inducing neurodegenerative disorder in a non-human animal, said pharmaceutical combination comprising 10.0-30.3 mg of KA per kg of the non-human and 0.25-1.4 mg of lorazepam per kg of the non-human.

In some aspects of the previous embodiments of a pharmaceutical combination for use in inducing neurodegenerative disorder in a non-human animal, the neurodegenerative disorder comprises epilepsy, brain injury, spinal cord injury, bipolar disorder, trigeminal neuralgia, attention-deficit hyperactivity disorder, partial seizures, adjunctive therapy for partial, myoclonic, tonic-clonic seizures, schizophrenia, neuropathic pain, seizures, Tourette syndrome, Alzheimer's disease, autism, anxiety disorder, mania, phantom limb syndrome, complex regional pain syndrome, paroxysmal extreme pain disorder, neuromyotonia, intermittent explosive disorder, borderline personality disorder, myotonia congenita, Frontotemporal dementia, multiple sclerosis, Amyotrophic Lateral Sclerosis, Parkinson's disease, and Huntington's disease, traumatic brain injury, and post-traumatic stress disorder. In certain aspects of the previous embodiments of a pharmaceutical combination for use in inducing neurodegenerative disorder in a non-human animal, the neurodegenerative disorder is epilepsy. In some aspects of the previous embodiments of a pharmaceutical combination for use in inducing neurodegenerative disorder in a non-human animal, the non-human animal does not exhibit status epilepticus and/or convulsive status epilepticus. In further aspects of the previous embodiments of a pharmaceutical combination for use in inducing neurodegenerative disorder in a non-human animal, the non-human animal exhibits hippocampal sclerosis and spontaneous hippocampal-onset seizures.

In other aspects of the previous embodiments of a pharmaceutical combination for use in inducing neurodegenerative disorder in a non-human animal, the administered pharmaceutical combination comprises a first composition comprising 10.0-30.3 mg of KA per kg of the non-human animal and a second composition comprising 0.25-1.4 mg of lorazepam per kg of the non-human animal. In further aspects of the previous embodiments of a pharmaceutical combination for use in inducing neurodegenerative disorder in a non-human animal, the first composition comprising 10.0-30.3 mg of KA per kg of the non-human and the second composition comprising 0.25-1.4 mg of lorazepam per kg of the non-human are formulated for subcutaneous injection. In even further aspects of the previous embodiments of a pharmaceutical combination for use in inducing neurodegenerative disorder in a non-human animal, the non-human animal is a rat.

Some embodiments of the present disclosure provide a method of inducing a neurodegenerative disorder in a non-human animal, said method comprising administering a pharmaceutical combination comprising 10.0-30.3 mg of KA per kg of the non-human animal and a 0.25-1.4 mg of lorazepam per kg of the non-human animal to the non-human animal, and further comprising inducing a neurodegenerative disorder in the non-human animal by said step of administering.

In some aspects of the previous embodiments of a method of inducing a neurodegenerative disorder in a non-human animal, the neurodegenerative disorder comprises epilepsy, brain injury, spinal cord injury, bipolar disorder, trigeminal neuralgia, attention-deficit hyperactivity disorder, partial seizures, adjunctive therapy for partial, myoclonic, tonic-clonic seizures, schizophrenia, neuropathic pain, seizures, Tourette syndrome, Alzheimer's disease, autism, anxiety disorder, mania, phantom limb syndrome, complex regional pain syndrome, paroxysmal extreme pain disorder, neuromyotonia, intermittent explosive disorder, borderline personality disorder, myotonia congenita, Frontotemporal dementia, multiple sclerosis, Amyotrophic Lateral Sclerosis, Parkinson's disease, and Huntington's disease, traumatic brain injury, and post-traumatic stress disorder. In certain aspects of the previous embodiments of a method of inducing a neurodegenerative disorder in a non-human animal, the neurodegenerative disorder is epilepsy. In some aspects of the previous embodiments of a method of inducing a neurodegenerative disorder in a non-human animal, the non-human animal does not exhibit status epilepticus and/or convulsive status epilepticus. In further aspects of the previous embodiments of a method of inducing a neurodegenerative disorder in a non-human animal, the non-human animal exhibits hippocampal sclerosis and spontaneous hippocampal-onset seizures.

In other aspects of the previous embodiments of a method of inducing a neurodegenerative disorder in a non-human animal, the administered pharmaceutical combination comprises a first composition comprising 10.0-30.3 mg of KA per kg of the non-human animal and a second composition comprising 0.25-1.4 mg of lorazepam per kg of the non-human animal. In further aspects of the previous embodiments of a method of inducing a neurodegenerative disorder in a non-human animal, the first composition comprising 10.0-30.3 mg of KA per kg of the non-human is administered subcutaneously in a single dose, and the second composition comprising 0.25-1.4 mg of lorazepam per kg of the non-human animal is administered subcutaneously in a single dose. In certain aspects of the previous embodiments of a method of inducing a neurodegenerative disorder in a non-human animal, the first composition comprising 10.0-30.3 mg of KA per kg of the non-human and the second composition comprising 0.25-1.4 mg of lorazepam per kg of the non-human are administered together in a single dosage form. In even further aspects of the previous embodiments of a method of inducing a neurodegenerative disorder in a non-human animal, the non-human animal is a rat.

Some embodiments provide a method of assaying the anti-neurodegenerative disorder efficacy of a compound or pharmaceutical composition, wherein said method comprises:

administering a compound or pharmaceutical composition postulated as having potential as an agent for treating a neurodegenerative disorder to a non-human animal which has been administered a pharmaceutical combination comprising 10.0-30.3 mg of KA per kg of the non-human animal and 0.25-1.4 mg of lorazepam per kg of the non-human animal, and

determining the rate of occurrence and/or severity of neuron degeneration induced in said non-human animal, wherein a decreased rate of occurrence and/or severity of neuron degeneration is associated with anti-neurodegenerative efficacy of the compound or pharmaceutical composition.

In some aspects of the previous embodiments of a method of assaying the anti-neurodegenerative disorder efficacy of a compound or pharmaceutical composition, the neurodegenerative disorder comprises epilepsy, brain injury, spinal cord injury, bipolar disorder, trigeminal neuralgia, attention-deficit hyperactivity disorder, partial seizures, adjunctive therapy for partial, myoclonic, tonic-clonic seizures, schizophrenia, neuropathic pain, seizures, Tourette syndrome, Alzheimer's disease, autism, anxiety disorder, mania, phantom limb syndrome, complex regional pain syndrome, paroxysmal extreme pain disorder, neuromyotonia, intermittent explosive disorder, borderline personality disorder, myotonia congenita, Frontotemporal dementia, multiple sclerosis, Amyotrophic Lateral Sclerosis, Parkinson's disease, and Huntington's disease, traumatic brain injury, and post-traumatic stress disorder. In certain aspects of the previous embodiments of a method of assaying the anti-neurodegenerative disorder efficacy of a compound or pharmaceutical composition, the neurodegenerative disorder is epilepsy. In some aspects of the previous embodiments of a method of assaying the anti-neurodegenerative disorder efficacy of a compound or pharmaceutical composition, the non-human animal does not exhibit status epilepticus and/or convulsive status epilepticus. In further aspects of the previous embodiments of a method of inducing a neurodegenerative disorder in a non-human animal, the non-human animal exhibits hippocampal sclerosis and spontaneous hippocampal-onset seizures.

In aspects of the previous embodiments of a method of assaying the anti-neurodegenerative disorder efficacy of a compound or pharmaceutical composition, the administered pharmaceutical combination comprises a first composition comprising 10.0-30.3 mg of KA per kg of the non-human animal and a second composition comprising 0.25-1.4 mg of lorazepam per kg of the non-human animal. In further aspects of the previous embodiments of a method of assaying the anti-neurodegenerative disorder efficacy of a compound or pharmaceutical composition, the first composition comprising 10.0-30.3 mg of KA per kg of the non-human is administered subcutaneously in a single dose, and the second composition comprising 0.25-1.4 mg of lorazepam per kg of the non-human animal is administered subcutaneously in a single dose. In certain aspects of the previous embodiments of a method of assaying the anti-neurodegenerative disorder efficacy of a compound or pharmaceutical composition, the first composition comprising 10.0-30.3 mg of KA per kg of the non-human and the second composition comprising 0.25-1.4 mg of lorazepam per kg of the non-human are administered together in a single dosage form. In even further aspects of the previous embodiments of a method of assaying the anti-neurodegenerative disorder efficacy of a compound or pharmaceutical composition, the non-human animal is a rat.

Some embodiments provide a kit comprising a single or separate dosage forms comprising 10.0-30.3 mg of kainic acid per kg of the non-human and 0.25-1.4 mg of lorazepam per kg of the non-human, wherein the dosage forms are co-presented in a same packaging or are separately packaged and available for sale together or independently of one another, and are co-marketed or co-promoted for co-administration.

These and additional aspects and features of the instant invention will be clarified by reference to the figures and detailed description set forth herein.

### BRIEF DESCRIPTION OF THE DRAWINGS

FIG. 1A-I depicts acute and chronic hippocampal neuropathology after systemic, concurrent administration of 15 mg/kg KA and lorazepam at either 3 mg/kg or 0.75 mg/kg. As shown in FIG. 1A-I, animals that received less lorazepam had more neurodegeneration and vice versa. FIG. 1A shows Fluoro-Jade B (FJB) staining; FIG. 1B depicts NeuN immunoreactivity; and FIG. 1C shows Timm staining in the dorsal hippocampus from an untreated control rat, demonstrating normal neuroanatomy. FIG. 1D shows FJB staining 4 days posttreatment (3 mg/kg lorazepam) showing no apparent neurodegeneration. FIG. 1E shows NeuN-immunostaining 10 weeks posttreatment (3 mg/kg lorazepam) exhibiting apparently normal neuroanatomy. FIG. 1F shows Timm staining 10 weeks posttreatment (1 mg lorazepam) and confirms normal granule cell efferents, that is, lack of mossy fiber sprouting. FIG. 1G shows FJB staining 4 days posttreatment (0.75 mg/kg lorazepam) showing widespread neurodegeneration in the dentate hilus, CA3, and CAL, while FIG. 1H shows NeuN-immunostaining 10 weeks posttreatment (0.75 mg/kg lorazepam) reveals extensive neuron loss in the dentate hilus, CA3, and CAI, that is, classic hippocampal sclerosis. FIG. 1I shows Timm staining 10 weeks posttreatment (0.75 mg/kg lorazepam), demonstrating aberrant reorganization of granule cell axons, that is, mossy fiber sprouting. (FIG. 1A-I scale bar is 200 μm).

FIG. 2A-D depicts continuous video-EEG monitoring of hippocampal seizures, including both kainate-induced (by systemic, concurrent administration of 15 mg/kg KA and lorazepam at either 3 mg/kg or 0.75 mg/kg) and spontaneous, recorded from the dentate gyrus in freely moving Sprague-Dawley rats. FIG. 2A depicts fifty-eight seconds of activity, recorded 44 min after kainate and lorazepam administration (15 mg/kg and 0.75 mg/kg, respectively). FIG. 2B depicts an eight hundred milliseconds extract from FIG. 2A, demonstrating epileptiform discharging of hippocampal granule cells. FIG. 2C depicts a rat's first spontaneous (focal) seizure 10 days post-kainate (15 mg/kg) and lorazepam (0.75 mg/kg) administration. The depicted trace represents 58 s of spontaneous activity. FIG. 2D depicts an eight hundred milliseconds extract from FIG. 2C, showing epileptiform discharging of hippocampal granule cells. Behavior during the spontaneous seizure was limited to staring; a few wet dog shakes were seen after the EEG signal returned to baseline. (FIG. 2A-D calibration bar: 2 mV in FIG. 2A-D; 4 s in FIG. 2A and FIG. 2C, 55 msec in FIG. 2B and FIG. 2D; sampling rate 2 kHz).

FIG. 3A-D depicts characteristics of spontaneous seizures after systemic, concurrent administration of 15 mg/kg KA and 0.75 mg/kg lorazepam. FIG. 3A depicts latency from treatment to the first spontaneous seizure as determined by continuous video-EEG recording with electrodes located in the dorsal dentate gyrus. The mean time to epilepsy was 12.1±(standard deviation) 1.7 days. FIG. 3B depicts the frequency of spontaneous seizures. Animals exhibited an average of 7.8±5.1 seizures per day during the first 2 weeks of spontaneous epilepsy. FIG. 3C depicts the distribution of seizures during the day (6:00-17:59) and night (18:00-5:59). A majority of seizures (72%) occurred during the day. FIG. 3D depicts seizure behavior. All spontaneous seizures that occurred during the first 2 weeks posttreatment were nonconvulsive. Starting at week 3, convulsive motor seizures were seen. Data for FIG. 3A-D are presented as mean±SEM; stages in FIG. 3B are according to the Racine scale (see for example, Racine R J. Modification of seizure activity by electrical stimulation: II. Motor seizure. Electroencephalogr Clin Neurophysiol 1972; 32:281-294, which is hereby incorporated by reference in its entirety).

FIG. 4A-B depicts the quantification of morphologic changes in the hippocampus at least 10 weeks after systemic, concurrent administration of 15 mg/kg kainate and lorazepam at either 3 mg/kg (middle bar of each subgroup Whole himmocampus, DG, CA, and IML) or 0.75 mg/kg (right bar of each subgroup Whole himmocampus, DG, CA, and IML), compared with age-match naïve rats (left bar of each subgroup Whole himmocampus, DG, CA, and IML). FIG. 4A depicts the hippocampal area, also subdivided into dentate gyrus (DG) and cornu ammonis (CA) subfields, relative to control, obtained from NeuN-immunostained or Nissl-stained brain sections. Three out of four hippocampi in the 0.75 mg/kg lorazepam treatment group exhibited DG hypertrophy. FIG. 4B depicts mean gray values obtained from the inner molecular layer (IML) in Timm-stained brain sections. Larger numbers indicate darker gray values, i.e. more mossy fiber sprouting into the IML. Data for FIG. 4A-B are presented as mean±SEM, with statistical analysis performed using student's t-test. For FIG. 4A-B, n=18-20 sections total from 4 brains for each group, with all P-values ≤0.01.

### DETAILED DESCRIPTION

The following description of particular aspect(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only. While the processes and compositions are described as using specific a specific order of individual steps or specific materials, it is appreciated that steps or materials may be interchangeable such that the description of the invention may include multiple steps or parts arranged in many ways as is readily appreciated by one of skill in the art.

The terminology used herein is for describing particular embodiments/aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

The presently-disclosed data demonstrates that a single dose of KA administered concurrently with a low dose of lorazepam can be used to dependably reproduce fundamental characteristics of acquired human TLE in non-human animals, while avoiding SE and/or cSE and its associated problems, for example, significant variability and mortality. More particularly, the present results demonstrate that 10.0-30.3 mg/kg of KA administered concurrently with 0.25-1.4 mg/kg of lorazepam can be used to dependably reproduce fundamental characteristics of acquired mesial TLE in rats, while avoiding cSE and its inherent problems. In some aspects, administering a dosage form of 0.25-1.4 mg/kg lorazepam and 10.0-30.3 mg/kg KA to a non-human animal blocks SE and/or cSE, but not acute hippocampal seizures (that persist for 3-4 h and are self-terminating), neurodegeneration, or epileptogenesis. In some aspects, animals receive single, simultaneous, subcutaneous injections of KA and lorazepam, which was effective in all animals, no additional attention is necessary, and there is no need to titrate dosing for each individual animal. This is unlike cSE-based models that often require multiple injections and/or substantial palliative care. The present results reveal that the following further advantages: a simple protocol, acute hippocampal seizures that persist for 3-4 h and are self-terminating, substantial hippocampal neurodegeneration, spontaneous hippocampal seizures after a 10-15 day seizure-free latent period, and a lack of both morbidity and mortality.

Furthermore, the presently-disclosed data demonstrates that the crux of animal models for neurodegenerative disorders, in particular epilepsy and/or TLE, should not be the induction of cSE, but rather of prolonged electrographic seizure activity, since seizures do not always have a significant behavioral component. In fact, human status epilepticus is often nonconvulsive. Along these lines, terminating SE, both in the laboratory and clinic, requires both adequate treatment and EEG confirmation that seizures have stopped.

Finally, the presently-disclosed data demonstrates that the non-human animal model of present disclosure can be used in order to (1) reveal new and different targets for intervention and (2) discover treatments that exploit these novel mechanisms. Despite the advantages of newer antiseizure drugs in the management of epilepsy, such as fewer adverse drug interactions or hypersensitivity reactions, their efficacy and tolerability has not improved much over the last 25 years. Consequently, −30% of patients with epilepsy do not respond satisfactorily to drug therapy, a figure that has also not budged during this time. One reason for this persistent problem is that, with very few exceptions, the same animal models have discovered all antiseizure drugs. Therefore, the present results reveal that the present disclosure provides a non-human animal model for use in the drug-screening repertoire, in an effort to discover substances targeting novel epileptogenic (the development of epilepsy) and ictogenic (manifestation of individual seizures) mechanisms. The present model is of particular use in drug discovery efforts focused on refractory TLE and neurodegeneration.

Accordingly, reference will now be made in detail to various embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits.

In various embodiments, an animal model in which non-human animals have been administered a pharmaceutical combination comprising a first composition comprising a glutamate receptor agonist and a second composition comprising a therapeutic agent, wherein the non-human animals exhibits a neurodegenerative disorder by said administration, are provided.

In other embodiments, pharmaceutical combinations for use in inducing a neurodegenerative disorder in a non-human animal, said pharmaceutical combinations comprising a first composition comprising a glutamate receptor agonist and a second composition comprising a therapeutic agent, are provided.

In further embodiments, methods for inducing a neurodegenerative disorder in a non-human animal, said methods comprising administering a pharmaceutical combination comprising a first composition comprising a glutamate receptor agonist and a second composition comprising a therapeutic agent to the non-human animal, and inducing a neurodegenerative disorder in the non-human animal by said step of administering, are provided.

In various embodiments, method of assaying the anti-neurodegenerative disorder efficacy of a compound or pharmaceutical composition, said methods comprising administering a compound or pharmaceutical composition postulated as having potential as an agent for treating a neurodegenerative disorder to a non-human animal which has been administered a pharmaceutical combination comprising a first composition comprising a glutamate receptor agonist and a second composition comprising a therapeutic agent, and determining the rate of occurrence and/or severity of neuron degeneration induced in said non-human animal, wherein a decreased rate of occurrence and/or severity of neuron degeneration is associated with anti-neurodegenerative efficacy of the compound or pharmaceutical composition, are provided.

In other embodiments, a kit comprising a single or separate dosage forms comprising a first composition comprising a glutamate receptor agonist and a second composition comprising a therapeutic agent, wherein the single or separate dosage forms are co-presented in a same packaging or are separately packaged and available for sale together or independently of one another, and are co-marketed or co-promoted for co-administration, are provided.

Accordingly, in various embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, and methods of assaying the anti-neurodegenerative disorder efficacy of a compound or pharmaceutical composition, a pharmaceutical combination (e.g. pharmaceutical composition) comprises a first composition comprising a glutamate receptor agonist and a second composition comprising a therapeutic agent. In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, a non-human animal exhibits a neurodegenerative disorder by administration of the pharmaceutical combination.

As used herein, the term “non-human animal” refers to all non-transgenic and transgenic non-human vertebrates, e.g., mammals and non-mammals, such as non-human primates, sheep, dogs, cats, horses, cows, rodents, amphibians, reptiles, etc. Optionally, in certain aspects of the various embodiments described herein, the non-human animal according to the present invention is non-transgenic and optionally is a non-transgenic rodent. Optionally, in particular aspects of the various embodiments described herein, the rodent is a rat. Optionally, in more particular aspects of the various embodiments described herein, the non-human animal is a non-transgenic Sprague-Dawley rat.

As used herein, the term “pharmaceutical combination” refers to simultaneously, separately, and/or sequentially administered one or more glutamate receptor agonists and one or more therapeutic agents. For example, in some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, a first compositions comprising one or more glutamate receptor agonists and a second composition comprising one or more therapeutic agents can be part of a single pharmaceutical composition (for example, for administration in a single dosage form) or can be in separate pharmaceutical compositions (for example, for administration in separate dosage forms, either simultaneously or sequentially). Thus, the term “pharmaceutical combination” includes a pharmaceutical composition. The term “component” refers to an active ingredient in the pharmaceutical combinations according to the present invention (for example, a glutamate receptor agonist, a therapeutic agent). The components of a pharmaceutical combination can be dosed independently or by use of different fixed combinations with distinguished amounts of the components, i.e., simultaneously or at different time points. The components of the pharmaceutical combination can then be administered, e.g. but not limited to, simultaneously or chronologically, that is, at different time points and with equal or different time intervals for any component.

As used herein, the term “glutamate receptor” refers to any receptor that binds and is activated by the neurotransmitter glutamate. Glutamate receptors can be divided into two groups: ionotropic glutamate receptors and Metabotropic glutamate receptors. Ionotropic glutamate receptors include Kainate, NMDA, and AMPA receptors. Metabotropic glutamate receptors (mGluR) indirectly activate ion-channels on the plasma membrane through a signaling cascade that involves G proteins. Optionally, in aspects the glutamate receptor of the present invention is Kainate receptor. Optionally, in aspects the glutamate receptor of the present invention is NMDA receptor. Optionally, in aspects the glutamate receptor of the present invention is AMPA receptor.

As used herein, the term “glutamate receptor agonist” refers to any glutamate receptor agonist (direct agonist or allosteric agonist), and includes any chemical entity that, upon administration to a non-human, results in activation or up-regulation of a biological activity associated with activation of the glutamate receptors in the non-human, including any of the downstream biological effects otherwise resulting from the binding to glutamate receptor of its natural ligand (glutamic acid). The glutamate receptor agonists include any agent that can induce glutamate receptor activation or any of the downstream biological effects of glutamate receptor activation.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the glutamate receptor agonist of present invention is, for example, kainic acid (KA), ibotenic acid, domoic acid, quisqualic acid, N-methyl-D-aspartic acid or N-methyl-D-aspartate (NMDA), a-amino-3-hydroxy-5-methyl-4-isoxazolepropionic acid (AMPA), L-2-amino-4 phosphonobutyric acid (L-AP4), 1-amino-1,3-dicarboxycyclopentane (ACPD), or any combination thereof.

As used herein, the term “therapeutic agent” refers to a drug, molecule, nucleic acid, protein, or any combinations thereof, or other substance that is neuroprotective and/or anti-neurodegenerative and/or anti-epileptogenic substances and/or substances used for the purposes of treating neurodegenerative disorders, in particular, epilepsy. As used herein the term “anti-epileptogenic” therapeutic agent refers to a therapeutic agent which is capable of inhibiting epileptogenesis (e.g., the development of epilepsy) when the agent is administered to a subject (e.g., a non-human animal).

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. Accordingly, these terms include preventing or reducing the frequency, severity, and/or duration of seizures in a subject.

In certain embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent of the present invention is one or more GABA modulators, prodrugs thereof, and pharmaceutically acceptable salts of the GABA modulators and prodrugs thereof.

As used herein, the term “GABA” is synonymous with the term “gamma-aminobutyric acid.” These terms may be used interchangeably.

As used herein, the term “GABA modulator” refers to a compound that either is structurally related to the neurotransmitter GABA but does not interact with the GABA receptor, or interacts with the GABA receptors, or is converted metabolically into GABA or a GABA agonist; or is an inhibitor of GABA uptake or degradation; or is a GABA receptor subtype-selective antagonist and/or agonist. This definition includes pharmaceutically acceptable salts, prodrugs or pharmaceutically acceptable salts of said prodrugs. The GABA modulator of present invention includes benzodiazepines, prodrugs thereof and pharmaceutically acceptable salts of the benzodiazepines and prodrugs thereof.

As used herein, the term “a benzodiazepine” refers to benzodiazepines as well as derivatives thereof, which are themselves normally classified as benzodiazepines. The term benzodiazepine also refers to benzodiazepine receptor subtype compounds as well as pharmaceutically acceptable salts of benzodiazepines, prodrugs of benzodiazepines and pharmaceutically acceptable salts of benzodiazepine prodrugs.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the benzodiazepines include, but are not limited to, lorazepam, muscimol, progabide, riluzole, baclofen, gabapentin, vigabatrin, tiagabine, lamotrigine, pregabalin, topiramate, diazepam, clonazepam, oxazepam, dipotassium chlorazepate, chlorazepate, chlordiazepoxide, mediazepam, flurazepam, clobasam, nitrasepam, flunitrasepam, astazolam, bromazepam, alprazolam, lormetasepam, temazepam, brotizolam, triazolam, chlorodiazepam, halazepam, prazepam, felbamate, nemotrizine, nemotrizine or pharmaceutically acceptable salts or prodrugs thereof or pharmaceutically acceptable salts of said prodrugs.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the GABA modulator is lorazepam, a prodrug thereof, a pharmaceutically acceptable salt of lorazepam, or a prodrug of a pharmaceutically acceptable salt of lorazepam.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the GABA modulators include, but are not limited to, muscimol, progabide, riluzole, baclofen, gabapentin (Neurontin®), vigabatrin, valproic acid, tiagabine (Gabitril®), lamotrigine (Lamictal®), pregabalin, phenyloin (Dilantin®), carbamazepine (Tegretol®), topiramate (Topamax®), prodrugs thereof and pharmaceutically acceptable salts of the GABA modulators prodrugs.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent is one or more anticonvulsant, prodrugs thereof, pharmaceutically acceptable salts of the anticonvulsants, or pharmaceutically acceptable salts of the anticonvulsants prodrugs.

As used herein, the term “anticonvulsant” therapeutic agent refers to a therapeutic agent capable of inhibiting (e.g., preventing, slowing, halting, or reversing) ictogenesis (e.g., seizure genesis) when the therapeutic agent is administered to a non-human.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the anticonvulsants include, but are not limited to: barbiturates, urethane, hydantoins such as phenyloin (Dilantin®), mephenyloin (Mesantoin®); succinimides such as ethosuximide (Zarontin®), oxazolidinediones such as trimethadione (Tridione®), carbamazepine (Tegretol®), primadone (Mysoline0), valproic acid (Depakote®), prodrugs thereof and pharmaceutically acceptable salts of the anticonvulsants and prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes one or more N-methyl-D-aspartate (hereinafter designated as “NMDA”) receptor antagonist, prodrugs thereof, and pharmaceutically acceptable salts of the NMDA antagonist, and prodrugs thereof. In aspects, the NMDA receptor antagonists of present invention include, for example, morphinans such as dextromethorphan or dextrorphan, ketamine, d-methadone. It also includes therapeutic agents that block a major intracellular consequence of NMDA-receptor activation, e.g. a ganglioside such as GM1 or GT1b, a phenothiazine such as trifluoperazine, or a naphthalenesulfonamide such as N-(6-aminothexyl)-5-chloro-1-naphthalenesulfonamide.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes one or more AMPA receptor antagonists, prodrugs thereof, pharmaceutically acceptable salts of the AMPA antagonist receptor and prodrugs thereof is provided.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes one or more agonist of CB1, CB2, and 5-HT1a receptors, and an allosteric modulator of μ and δ-opioid receptors.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes Cannabidiol, prodrugs thereof, and pharmaceutically acceptable salts of the Cannabidiol and prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent inhibits mixed lineage kinase family, e.g. c-jun N-terminal kinase (JNK). Optionally, the therapeutic agent is CEP-1347. CEP-1347 is disclosed, for example, in J Biol Chem. 2001 Jul. 6; 276(27):253028. Epub 2001 Apr. 26, Cep-1347 (KT7515), a semisynthetic inhibitor of the mixed lineage kinase family by Maroney A C et al.; herein incorporated by reference in its entirety. In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent is CEP-1347, prodrugs thereof, and pharmaceutically acceptable salts of the CEP-1347 and prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes a gamma-secretase modulator and anti-inflammatory, latter through acting on microglia. Optionally, in some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes CHF 5074. CHF 5074 is disclosed, for example, in Sivilia S et al. Multi-target action of the novel anti-Alzheimer compound CHF5074: in vivo study of long term treatment in Tg2576 mice. BMC Neurosci. 2013; 14:44; herein incorporated by reference in its entirety. In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes CHF 5074, prodrugs thereof, and pharmaceutically acceptable salts of the CHF 5074 and prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent chelates iron, e.g. Deferiprone. In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes Deferiprone, prodrugs thereof, and pharmaceutically acceptable salts of the Deferiprone and prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes a neuronal potassium channel opener, e.g. Flupirtine. In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes Flupirtine, prodrugs thereof, and pharmaceutically acceptable salts of the Flupirtine and prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent factivates voltage-gated potassium channels, e.g. Retigabine or ezogabine. In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes Retigabine or ezogabine, prodrugs thereof, and pharmaceutically acceptable salts of the Retigabine or ezogabine and prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes any combination of two or more of the following: GABA modulators, prodrugs thereof, and pharmaceutically acceptable salts of the GABA modulators and prodrugs thereof; anticonvulsants, prodrugs thereof, and pharmaceutically acceptable salts of the anticonvulsants and prodrugs thereof; NMDA receptor antagonists, prodrugs thereof, and pharmaceutically acceptable salts of the NMDA antagonist receptors and prodrugs thereof; AMPA receptor antagonists, prodrugs thereof, pharmaceutically acceptable salts of the AMPA antagonist receptor and prodrugs thereof; agonists of CB 1, CB2, and 5-HTla receptors, and an allosteric modulator of II and 6-opioid receptors; therapeutic agents that inhibits mixed lineage kinase family; gamma-secretase modulators and anti-inflammatory; therapeutic agents that chelates iron; a neuronal potassium channel openers; and voltage-gated potassium channels activators.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the therapeutic agent includes any combination of two or more of the following: Lorazepam, prodrugs thereof, and pharmaceutically acceptable salts of the Lorazepam and prodrugs thereof; Cannabidiol, prodrugs thereof, and pharmaceutically acceptable salts of the Cannabidiol and prodrugs thereof; CEP-1347, prodrugs thereof, and pharmaceutically acceptable salts of the CEP-1347 and prodrugs thereof; CHF 5074, prodrugs thereof, and pharmaceutically acceptable salts of the CHF 5074 and prodrugs thereof; Deferiprone, prodrugs thereof, and pharmaceutically acceptable salts of the Deferiprone and prodrugs thereof; Flupirtine, prodrugs thereof, and pharmaceutically acceptable salts of the Flupirtine and prodrugs thereof; and Retigabine or ezogabine, prodrugs thereof, and pharmaceutically acceptable salts of the Retigabine or ezogabine and prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, a pharmaceutical combination (e.g. pharmaceutical composition) includes: a) KA, Ibotenic acid, domoic acid, Quisqualic acid, NMDA, AMPA, L-AP4, or ACPD, prodrugs thereof, pharmaceutically acceptable salts and prodrugs thereof, or any combinations thereof; and b) Lorazepam, Cannabidiol, CEP-1347, CHF 5074, Deferiprone, Flupirtine, Retigabine, or ezogabine, prodrugs thereof, pharmaceutically acceptable salts and prodrugs thereof, or any combinations thereof.

In some embodiments of the instantly-disclosed non-human animals models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises: a) KA, prodrugs thereof, pharmaceutically acceptable salts of KA and/or prodrugs thereof; and b) lorazepam, prodrugs thereof, pharmaceutically acceptable salts of lorazepam and/or prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises: a) about 10.0-30.3 mg/kg, about 10.0-25.0 mg/kg, about 13.2-30.3 mg/kg, about 13.2-25.0 mg/kg, about 13.2-15.1 mg/kg, optionally about 10.0 mg/kg, about 13.2 mg/kg, about 13.3 mg/kg, about 14 mg/kg, about 14.2 mg/kg, about 14.7 mg/kg, about 15 mg/kg, about 15.1 mg/kg, about 20.0 mg/kg, about 25.0 mg/kg, about 30.0 mg/kg, or about 30.3 mg/kg, and any value or range between about 10.0-30.3 mg/kg KA, prodrugs thereof, pharmaceutically acceptable salts of KA, and/or prodrugs thereof (wherein kg is the weight of the non-human animal); and b) about 0.25-1.4 mg/kg, 0.6-1.4 mg/kg, about 0.25 mg/kg, about 0.5 mg/kg, about 0.6 mg/kg, about 0.7 mg/kg, about 0.75 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, or any value or range between about 0.25-1.4 mg/kg lorazepam, prodrugs thereof, pharmaceutically acceptable salts of lorazepam, and/or prodrugs thereof (wherein kg is the weight of the nonhuman animal). In certain embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises the effective dosage combinations of KA, prodrugs thereof, pharmaceutically acceptable salts of KA, and/or prodrugs thereof and lorazepam, prodrugs thereof, pharmaceutically acceptable salts of lorazepam, and/or prodrugs thereof listed in Table 1.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination (e.g. pharmaceutical composition) comprises: a) about 15.0 mg/kg KA, prodrugs thereof, pharmaceutically acceptable salts of KA, and/or prodrugs thereof (kg is the weight of the non-human animal); and b) about 0.75 mg/kg lorazepam, prodrugs thereof, pharmaceutically acceptable salts of lorazepam, and/or prodrugs thereof (kg is the weight of the non-human animal).

As used herein, the term “prodrug” refers to compounds that are drug precursors which, following administration, release the drug in vivo via a chemical or physiological process (e.g., a prodrug on being brought to the physiological pH or through enzyme action is converted to the desired drug form). “Prodrugs” are intended to include any covalently bonded carriers that release an active parent drug of the present invention in vivo when such prodrug is administered to a subject, including a non-human animal.

As used herein, the term “pharmaceutically acceptable salt” of a compound means a salt that is pharmaceutically acceptable and that possesses the desired pharmacological activity of the parent compound. Pharmaceutically acceptable salts include salts of the parent compounds that are prepared with relatively nontoxic acids or bases, wherein such compounds are modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucohep tonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

It should be understood that all references to the presently-disclosed glutamate receptor agonists (e.g., but not limited to, KA), therapeutic agents (e.g., but not limited to, lorazepam), pharmaceutically acceptable salts thereof, and/or prodrugs thereof include solvent addition forms (solvates) or crystal forms (polymorphs) as defined herein.

As used herein, the terms “crystal polymorphs” or “polymorphs” or “crystal forms” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of compounds can be prepared by crystallization under different conditions. Additionally, the compounds of the present invention, for example, glutamate receptor agonists (e.g., but not limited to, KA), therapeutic agents (e.g., but not limited to, lorazepam), pharmaceutically acceptable salts thereof, and/or prodrugs thereof, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, “solvates” means solvent addition forms that contain either stoichiometric or non stoichiometric amounts of solvent. Some compounds or salts have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

As used herein, the term “about” in relation to a numerical value x, where used in the description and the appendant claims means, for example, x+/−10%.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, after the administration of the pharmaceutical combination according to any of the preceding embodiments, the non-human animal exhibits acute hippocampal seizures, neurodegeneration, and/or epileptogenesis; optionally, the non-human animal does not exhibit SE and/or cSE; optionally, the non-human animal is free of SE and/or cSE and exhibits hippocampal seizures, neurodegeneration, or epileptogenesis; optionally, the non-human animal exhibits characteristics of acquired human epilepsy, optionally TLE; optionally, the non-human animal exhibits characteristics of human mesial TLE classified as International League Against Epilepsy [ILAE] Type I) (see, for example, Ingmar Bluemcke et al. Epilepsia, 57(3):348-358, 2016, incorporated by reference in its entirety); optionally, the non-human animal exhibits hippocampal seizures that persist for 3-4 h and are self-terminating; optionally, the non-human animal exhibits spontaneous hippocampal seizures after a 10-15 day seizure-free period, and optionally, lacks both morbidity and mortality. In some embodiments, the non-human animal exhibits one or combination of two or more of any of the above-mentioned characteristics.

As used herein, the term “administering” or the like refers and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Optionally, the route of administration according to some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits is subcutaneous or intraperitoneal. Optionally, the route of administration according to some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits is subcutaneous injection.

As used herein, the term “disease” or “condition” or “disorder” refers to a state of being or health status of a non-human animal or human capable of being treated with anti-neurodegenerative or anti-epileptogenic substances.

As used herein, the term “neurodegenerative disorder” includes any disorder characterized by neural damage and includes but is not limited to epilepsy, brain injury, spinal cord injury, bipolar disorder, trigeminal neuralgia, attention-deficit hyperactivity disorder, partial seizures, adjunctive therapy for partial, myoclonic, tonic-clonic seizures, schizophrenia, neuropathic pain, seizures, Tourette syndrome, Alzheimer's disease, autism, anxiety disorder, mania, phantom limb syndrome, complex regional pain syndrome, paroxysmal extreme pain disorder, neuromyotonia, intermittent explosive disorder, borderline personality disorder, myotonia congenita, Frontotemporal dementia, multiple sclerosis, Amyotrophic Lateral Sclerosis, Parkinson's disease, and Huntington's disease, traumatic brain injury, and post-traumatic stress disorder.

As used herein, the terms “epileptic disorder,” “epilepsy disorder,” “seizure disorder,” or “epilepsy” include a spectrum of chronic neurological disorders most often characterized by the presence of unprovoked seizures. Epilepsy as used herein, includes injury to the brain (e.g. from trauma, stroke, or cancer) or genetic mutation. Non-human animals experiencing two or more unprovoked seizures may be considered to have epilepsy.

Types of epilepsy disorders according to some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits include, for example, benign Rolandic epilepsy, frontal lobe epilepsy, temporal lope epilepsy (TLE); infantile spasms, juvenile myoclonic epilepsy, juvenile absence epilepsy, West Syndrome, childhood absence epilepsy (e.g. pyknolepsy), febrile seizures, progressive myoclonus epilepsy of Lafora, Lennox-Gastaut syndrome, Landau-Kleffner syndrome, Dravet syndrome, Generalized Epilepsy with Febrile Seizures, Severe Myoclonic Epilepsy of Infancy, Unverricht-Lundborg disease, Benign Neonatal Familial Convulsions, early myoclonic encephalopathies, migrating partial epilepsy, infantile epileptic encephalopathies, Tuberous Sclerosis Complex, focal cortical dysplasia, Miller-Dieker Syndrome, Angelman's syndrome, Fragile X syndrome, Ohtahara Syndrome, epilepsy in autism spectrum disorders, subcortical band heterotopia, Type I Lissencephaly, Walker-Warburg syndrome, Alzheimer's disease, posttraumatic epilepsy, progressive myoclonus epilepsies, reflex epilepsy, Rasmussen's syndrome, temporal lobe epilepsy, limbic epilepsy, status epilepticus, abdominal epilepsy, massive bilateral myoclonus, catamenial epilepsy, photosensitive epilepsy, or Jacksonian seizure disorder.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises a separate dosage form comprising (a) a first composition comprising a glutamate receptor agonist according to any of the preceding embodiments and (b) a second composition comprising the therapeutic agent according to any of the preceding embodiments. Optionally, the compositions (a) and (b) are provided in distinct preparations, i.e. in separate dosage forms. Optionally, the compositions (a) and (b) are administered simultaneously (co-administered) or subsequently. Optionally, the compositions (a) and (b) are provided in a single dosage form.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the separate dosage forms are co-presented in a same packaging or kit, or are separately packaged and available for sale independently of one another, but are co-marketed or co-promoted for simultaneous and/or subsequent administration, in particular for use in inducing acute hippocampal seizures, neurodegeneration, and/or epilepsy in a non-human and/or for use in screening or assaying an anti-neurodegenerative disorder of a compound or pharmaceutical composition and/or therapies.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination of the present invention comprises a single dosage form comprising the glutamate receptor agonist according to any of the preceding embodiments and the therapeutic agent according to any of the preceding embodiments.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the single dosage form is marketed or promoted for use in inducing acute hippocampal seizures, neurodegeneration, and/or epilepsy in a non-human and/or for use in screening or assaying an anti-neurodegenerative disorder of a compound or pharmaceutical composition and/or anti-neurodegenerative disorder therapies.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the single dosage form includes mixing a therapeutic effective amount of the glutamate receptor agonist according to any of the preceding embodiments and a therapeutic effective amount of the therapeutic agent according to any of the preceding embodiments prior to administration to the non-human.

The selection of the dosage of the glutamate receptor agonists and the therapeutic agents according to any of the preceding embodiments is sufficient to induce conditions according to any of the preceding embodiments, in particular, acute hippocampal seizures, neurodegeneration, and/or epilepsy in a non-human.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises a separate dosage form comprising (a) a first composition comprising KA and (b) a second composition comprising one or more therapeutic agents according to any of the preceding embodiments.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises a separate dosage form comprising (a) a first composition comprising KA and (b) a second composition comprising a benzodiazepine.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises a separate dosage form comprising (a) a first composition comprising KA and (b) a second composition comprising lorazepam.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises a separate dosage form comprising (a) a first composition comprising about 10.0-30.3 mg/kg, about 10.0-25.0 mg/kg, about 13.2-30.3 mg/kg, about 13.2-25 mg/kg, about 13.2-15.1 mg/kg, optionally about 10.0 mg/kg, about 13.2 mg/kg, about 13.3 mg/kg, about 14.0 mg/kg, about 14.2 mg/kg, about 14.7 mg/kg, about 15.0 mg/kg, about 15.1 mg/kg, about 20.0 mg/kg, about 25.0 mg/kg, or about 30.3 mg/kg, and any value or range between about 10.0-30.3 mg/kg KA, prodrugs thereof, pharmaceutically acceptable salts of KA, and/or prodrugs thereof (wherein kg is the weight of the non-human animal); and (b) a second composition comprising about 0.25-1.4 mg/kg, 0.6-1.4 mg/kg, about 0.25 mg/kg, about 0.5 mg/kg, about 0.6 mg/kg, about 0.7 mg/kg, about 0.75 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, or any value or range between about 0.25-1.4 mg/kg lorazepam, prodrugs thereof, pharmaceutically acceptable salts of lorazepam, and/or prodrugs thereof (wherein kg is the weight of the nonhuman animal). In certain embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises a separate dosage form comprising of (a) KA, prodrugs thereof, pharmaceutically acceptable salts of KA, and/or prodrugs thereof and (b) lorazepam, prodrugs thereof, pharmaceutically acceptable salts of lorazepam, and/or prodrugs thereof as listed in Table 1.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, a pharmaceutical combination for use in inducing neurodegenerative disorders, epileptic seizures or similar disorders in a non-human, comprising a single dosage form comprising a KA and one or more of the therapeutic agents according to any of the preceding embodiments.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises a single dosage form comprising (a) a first composition comprising about 10.0-30.3 mg/kg, about 10.0-25.0 mg/kg, about 13.2-30.3 mg/kg, about 13.2-25 mg/kg, about 13.2-15.1 mg/kg, optionally about 10.0 mg/kg, about 13.2 mg/kg, about 13.3 mg/kg, about 14 mg/kg, about 14.2 mg/kg, about 14.7 mg/kg, about 15.0 mg/kg, about 15.1 mg/kg, about 20.0 mg/kg, about 25 mg/kg, or about 30.3 mg/kg, and any value or range between about 10.0-30.3 mg/kg KA, prodrugs thereof, pharmaceutically acceptable salts of KA, and/or prodrugs thereof (wherein kg is the weight of the non-human animal); and (b) a second composition comprising about 0.25-1.4 mg/kg, 0.6-1.4 mg/kg, about 0.25 mg/kg, about 0.5 mg/kg, about 0.6 mg/kg, about 0.7 mg/kg, about 0.75 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, or any value or range between about 0.25-1.4 mg/kg lorazepam, prodrugs thereof, pharmaceutically acceptable salts of lorazepam, and/or prodrugs thereof (wherein kg is the weight of the nonhuman animal).

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises a single dosage form comprising about 10-30.3 mg/kg of KA (depending on the non-human weight) and about 0.25-1.4 mg/kg of a benzodiazepine (depending on the non-human weight).

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination (e.g. pharmaceutical composition) comprises: a) KA, lbotenic acid, domoic acid, Quisqualic acid, NMDA, AMPA, L-AP4, or ACPD, prodrugs thereof, pharmaceutically acceptable salts and prodrugs thereof, or any combinations thereof; and b) Lorazepam, Cannabidiol, CEP-1347, CHF 5074, Deferiprone, Flupirtine, Retigabine, or ezogabine, prodrugs thereof, pharmaceutically acceptable salts and prodrugs thereof, or any combinations thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination (e.g. pharmaceutical composition) comprises: a) KA, prodrugs thereof, pharmaceutically acceptable salts and prodrugs thereof; and b) Lorazepam, prodrugs thereof, pharmaceutically acceptable salts and prodrugs thereof.

In some embodiments of the instantly-disclosed non-human animal models, pharmaceutical combinations or pharmaceutical compositions for use in inducing neurodegenerative disorder in a non-human animal, methods of inducing a neurodegenerative disorder in a non-human animal, methods of assaying the anti-neurodegenerative disorder efficacy of a compound or pharmaceutical composition, and kits, the pharmaceutical combination comprises a) about 10.0-30.3 mg/kg KA, prodrugs thereof, pharmaceutically acceptable salts and prodrugs thereof (kg is the weight of the non-human animal); and b) about 0.25-1.4 mg/kg Lorazepam, prodrugs thereof, pharmaceutically acceptable salts and prodrugs thereof (kg is the weight of the non-human animal).

As used herein, the term “dosage form” refers to the particular format of the pharmaceutical (e.g., the pharmaceutical combination as described herein), and depends on the route of administration. For example, a dosage form can be in a liquid, e.g., a saline solution for injection, capsules, tablets, pills, films, ointments, creams, solutions, suspensions, aerosols, pastes, drops, suppositories, powders for reconstitution, injectables, intravenous solutions and the like.

As used herein, the term “pharmaceutical combination” is intended to encompass a product comprising the components of present invention, and, optionally, the inert ingredient(s) (pharmaceutically acceptable excipients) that make up a carrier, as well as any product which results, directly or indirectly, from combination, complexation, or aggregation of any two or more of the components, or from dissociation of one or more of the ingredients, or from other types of reactions or interactions of one or more of the ingredients. Accordingly, the pharmaceutical combinations of the present invention encompass any composition made by admixing the compounds of present invention and, optionally, a pharmaceutically acceptable excipient.

In another aspect of the invention, a method of screening or assaying anti-neurodegenerative and/or anti-epileptic efficacy of a compound, pharmaceutical composition, or therapies is provided. For example, the method of present invention can be used to determine whether devices, methods, assays, antibodies have beneficial effects.

In one embodiment, the method comprises administering a compound or pharmaceutical composition used for treating neurodegenerative disorders, epilepsy or other seizure-related disorders to a non-animal, wherein the non-human has been administered with a pharmaceutical combination comprising the glutamate receptor agonists and the therapeutic agents according to any of the preceding embodiments, determining the condition of the disorder, optionally, the rate of occurrence and/or severity of neuron degeneration and/or any seizure induced in said non-human, wherein a decreased rate of occurrence and/or severity of neuron degeneration and/or seizure and/or improving the conditions of the disorder is associated with anti-neurodegenerative, anti-epileptic or related disorders efficacy of the compound or pharmaceutical composition, optionally, thereby an effective compound or pharmaceutical composition used for treating neurodegenerative disorders, epilepsy or other seizure-related disorders in humans can be selected.

In one embodiment, the method comprises administering a compound or pharmaceutical composition used for treating neurodegenerative disorders, epilepsy or other seizure-related disorders to a non-animal, wherein the non-human has been administered with a pharmaceutical combination comprising a) KA, prodrugs thereof, pharmaceutically acceptable salts and prodrugs thereof and b) lorazepam, prodrugs thereof, pharmaceutically acceptable salts and prodrugs thereof according to any of the preceding embodiments; determining the condition of the disorder, optionally, the rate of occurrence and/or severity of neuron degeneration and/or any seizure induced in said non-human, wherein a decreased rate of occurrence and/or severity of neuron degeneration and/or seizure and/or improving the conditions of the disorder is associated with anti-neurodegenerative, anti-epileptic or related disorders efficacy of the compound or pharmaceutical composition, optionally, thereby an effective compound or pharmaceutical composition used for treating neurodegenerative disorders, epilepsy or other seizure-related disorders in humans can be selected.

In another aspect of the invention, a kit for use in inducing seizures, neurodegeneration, and/or epilepsy in a non-human animal is provided.

In one embodiment, the kit comprises a pharmaceutical combination comprising one or more of the glutamate receptor agonists according to any of the preceding embodiments and one or more of the therapeutic agent according to any of the preceding embodiments.

In another embodiment, the kit comprises a pharmaceutical combination comprising KA and lorazepam according to any of the preceding embodiments. Optionally, the kit comprises a separate dosage forms comprising about 10.0-30.3 mg/kg of KA and about 0.25-1.4 mg/kg Lorazepam.

In one embodiment, the kit of present invention comprises separate dosage forms of the component of the pharmaceutical combination according to the present invention and any of the preceding embodiments, but are co-presented in a separate packaging or are separately packaged and available for sale independently of one another, but are co-marketed or co-promoted for simultaneous and/or subsequent administration, in particular for use in inducing acute hippocampal seizures, neurodegeneration, and/or epilepsy in a non-human and/or for use in screening or assaying an anti-neurodegenerative disorder of a compound or pharmaceutical composition.

### EXAMPLES

The following examples are given by way of illustration and are in no way intended to limit the scope of the present invention.

### Example 1

### Materials and Methods

Unless specified otherwise, the following experimental techniques were used in the Examples.

### Animals:

Male Sprague-Dawley rats (Harlan-Winkelmann, Borchen, Germany), weighing approximately 330 g (range 318-344 g), were treated in accordance with the guidelines of the European community (EUVD 86/609/EEC). Rats were housed in an on-site animal facility (21-25° C.; 31-47% humidity) under a 12:12 light/dark cycle with ad libitum access to food and water.

### Kainic Acid+Lorazepam Administration:

Various subcutaneous injections of kainic acid monohydrate (K0250, 10 mg/ml in phosphate-buffered saline; Sigma-Aldrich, Germany) and lorazepam (2 mg/ml; Pfizer, Germany) were administered under isoflurane sedation according to Table 1 and Table 2. Rats were placed in an acrylic box containing 5% isoflurane in oxygen until sedation was achieved (15-30 s), then removed and placed on a clean table where the injections were given. Following injections, rats were housed in clear acrylic boxes allowing free movement and visual observation.

TABLE 1
(Effective Dosage,
n ≥ 4 for all dosages)

| Kainic | Lorazepam |
|---|---|
| acid dose | dose |
| (mg/kg) | (mg/kg) |
| 10.0 | 0.25 |
| 13.3 | 0.25 |
| 15.0 | 0.25 |
| 10.0 | 0.50 |
| 14.0 | 0.50 |
| 15.0 | 0.50 |
| 13.3 | 0.6 |
| 14.0 | 0.6 |
| 14.2 | 0.6 |
| 15.1 | 0.6 |
| 10.0 | 0.75 |
| 13.3 | 0.75 |
| 14.0 | 0.75 |
| 14.7 | 0.75 |
| 15.0 | 0.75 |
| 15.1 | 0.75 |
| 20.0 | 0.75 |
| 25.0 | 0.75 |
| 30.0 | 0.75 |
| 20.0 | 1.0 |
| 13.2 | 1.3 |
| 25.0 | 1.3 |
| 14.2 | 1.4 |
| 15.1 | 1.4 |
| 30.3 | 1.4 |

(Ineffective Dosage,
Comparative study,
n ≥ 4 for all dosages)

| Kainic | Lorazepam |
|---|---|
| acid dose | dose |
| (mg/kg) | (mg/kg) |
| 10.0 | 1.5 |
| 10.0 | 2.8 |
| 15.9 | 3.1 |
| 17.0 | 3.1 |
| 15.9 | 3.4 |
| 17.0 | 3.4 |
| 15.1 | 3.8 |
| 15.0 | 4.4 |

### Seizure Monitoring (Continuous Video-EEG):

EEG data were acquired via either (1) recording electrodes with tips located in the dentate gyrus (approximate coordinates 2 mm lateral, 3 mm caudal to bregma, and 3.5 mm below the brain surface) or (2) screws with tips on the brain surface. Reference ground was always a screw located caudal and medial to the recording site and was not dorsal to the hippocampus. Electrodes and ground screws were connected to miniature wireless transmitters (FT20; Data Sciences International, U.S.A.) that were implanted subcutaneously on the animal's flank. All surgeries were performed in a stereotaxic apparatus (David Kopf) under isoflurane anesthesia (3-5% in oxygen). Spontaneous activity was recorded continuously (24/7) and stored digitally and automatically in 3-h epochs using LabChart 7 software (ADInstruments, New Zealand, as described in Norwood B A, Bumanglag A V, Osculati F, et al. Classic hippocampal sclerosis and hippocampal-onset epilepsy produced by a single “cryptic” episode of focal hippocampal excitation in awake rats. J Comp Neurol 2010; 518:3381-3407; and Harvey B D, Sloviter R S. Hippocampal granule cell activity and c-Fos expression during spontaneous seizures in awake, chronically epileptic, pilocarpine-treated rats: implications for hippocampal epileptagenesis. J Comp Neural 2005; 488:442463; herein incorporated by reference in their entirety). All files were evaluated by at least two experienced reviewers; at least one reviewer was blinded to the treatment. Recordings were assessed visually, and all events with amplitudes obviously larger than baseline were analyzed. Simultaneous video monitoring used Edimax IC-7110W infrared cameras (Taiwan). Video files were captured at 15 frames/s and time-stamped for integration with the EEG data using SecuritySpy surveillance software (Ben Software, United Kingdom) and stored digitally. Seizures were scored according to the Racine scale (Racine R J. Modification of seizure activity by electrical stimulation: IL Motor seizure. Electroencephalogr Clin Neurophysiol 1972; 32:281-294; herein incorporated by reference in its entirety).

### Perfusion Fixation:

Rats received an overdose of ketamine (>100 mg/kg, i.p.) and xylazine (10 mg/kg, i.p.) and were then gravity-perfused through the aorta with 0.9% saline for 90 s to remove intravascular blood. This was followed by 8 min of aortal perfusion with paraformaldehyde (4%) in 0.1 M phosphate buffer (pH 7.4). Brains were immediately removed from the skull and placed in 4% paraformaldehyde solution for at least 48 h before being sectioned (30 urn) on a freezing microtome.

### Fluorescence and Light Microscopy:

Nissl staining, Fluoro-Jade B staining, Timm staining, and neuronal nuclear antigen (NeuN) immunocytochemistry were performed on the resultant sections (as described in Norwood B A, Bumanglag A V, Osculati F, et al. Classic hippocampal sclerosis and hippocampal-onset epilepsy produced by a single “cryptic” episode of focal hippocampal excitation in awake rats. J Comp Neurol 2010; 518:3381-3407; herein incorporated by reference in its entirety). Images were acquired with a DMI6000B microscope equipped with a DCF360FX camera (Leica, Germany).

### Quantification of Neurodegeneration:

Fluoro-Jade B-positive neurons were counted in matching Fluoro-Jade B-stained sections from the dorsal hippocampus (one section per animal) using the Count Tool in Adobe Photoshop CS6.

### Quantification of Hippocampal Area:

The area of five matching, nonadjacent NeuN-immunostained or Nissl-stained sections from throughout the dorsal hippocampus was measured using the Adobe Photoshop CS6 Extended Measurement feature to calculate the area bounded by an irregular border (as described in Watkins J C, Evans R H. Excitatory amino acid transmitters. Annu Rev Pharmacol Toxicol 1981; 21:165-204; herein incorporated by reference in its entirety). Values were obtained for the entire hippocampus (excluding the fimbria), dentate gyrus, and cornu ammonis. Group means were compared using Student's t-test.

Quantification of Mossy Fiber Sprouting, that is, Timm Staining:

Five Timm-stained sections, equally distributed throughout the dorsal hippocampus, were evaluated using the Adobe Photoshop CS6 Histogram feature, which calculates the mean gray value for a selected area. Color images were converted to grayscale and inverted. The mean gray values for 64 pixel squares in the intermolecular layer were recorded and averaged. Background was calculated from a cell-free area in stratum radiatum and subtracted from the intermolecular layer values. Group means were compared using Student's t-test.

### Example 2

### Smaller Lorazepam Doses Increase Hippocampal Neurodegeneration

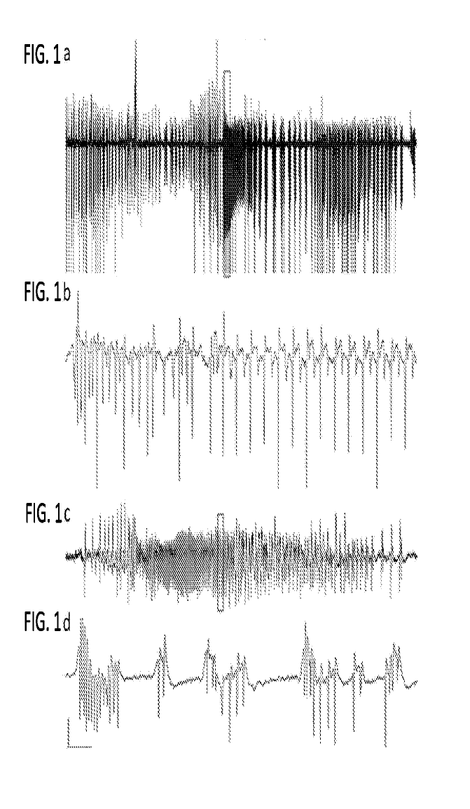

Various doses of lorazepam were evaluated (0.25-1.5 mg/animal; approximately 0.6-4.5 mg/kg, depending on weight), whereas the KA dose was kept constant (5 mg/animal; equivalent to 13.2-30.3 mg/kg, depending on weight). These doses of lorazepam are far below what is typically used to terminate experimental SE in rodents (6-8 mg/kg). Although acute hippocampal seizures were induced by KA at 3 mg/kg (n=8) and 4.5 mg/kg (n=5), neither neurodegeneration nor later spontaneous seizures were detected. Animals that received less lorazepam had more neurodegeneration and vice versa, as shown in FIG. 1A-I. By systematically reducing the lorazepam dose according to the present invention, it is found the optimal amount to be about 0.25-1.4 mg/kg, preferably, 0.6-0.8 mg/kg, more preferably about 0.75 mg/kg. An average of 565.4±(standard deviation) 43.7 Fluoro-Jade B-positive neurons were counted in dorsal hippocampus sections from animals that were sacrificed 4 days after receiving 15 mg/kg KA and about 0.75 mg/kg lorazepam, compared with 0.0±0.0 in animals that received 15 mg/kg KA and 3 mg/kg lorazepam. Broken down into hippocampal subfields, the mean values were 255.4±31.7 for CA1, 273.0±29.1 for CA3, and 37±7.7 for the hilus. During the 24 h immediately following KA and lorazepam administration, not a single animal in any group exhibited any convulsive seizures, let alone cSE, as determined by continuous video-EEG monitoring. At no time did any animal exhibit signs of morbidity; i.e. the survival rate was 100%.

### Example 3

### Low-Dose Lorazepam Blocks Kainic Acid-Induced Convulsive Seizures, but not Hippocampal Seizure Activity

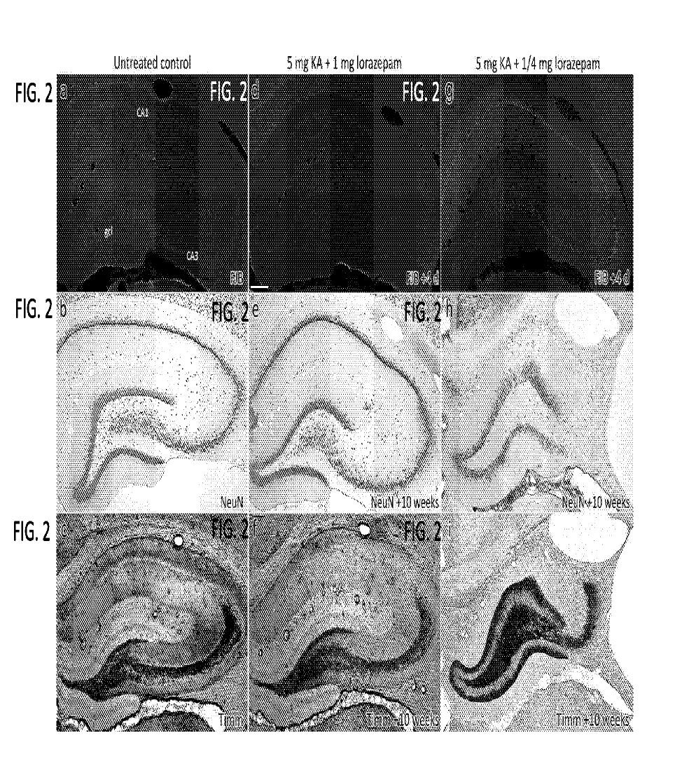

Following simultaneous, subcutaneous administration of 15 mg/kg KA and 0.75 mg/kg lorazepam, aberrant electrographic activity was detected within minutes and the first hippocampal seizures after 30-40 min, as determined with electrodes located in the dorsal dentate gyrus. Epileptiform discharging of hippocampal granule cells (FIG. 2A-D) persisted for at least 3 h in all animals, with an average of 3.3±0.4 h. During the treatment, seizure behavior was limited to occasional wet dog shakes, which were observed in some, but not all, rats. As seizures were self-terminating, no additional lorazepam was administered. On the 3 days following treatment, animals appeared and behaved normally. None presented with any sign of morbidity, for example, 10% weight loss, jumpiness, or reduced mobility. Consequently, none required palliative care. Animals that received 15 mg/kg KA and 3 mg/kg lorazepam exhibited an average of 12±7 min of hippocampal seizures.

### Example 4

### Spontaneous Hippocampal Seizures Arise after a Discrete Latent Period

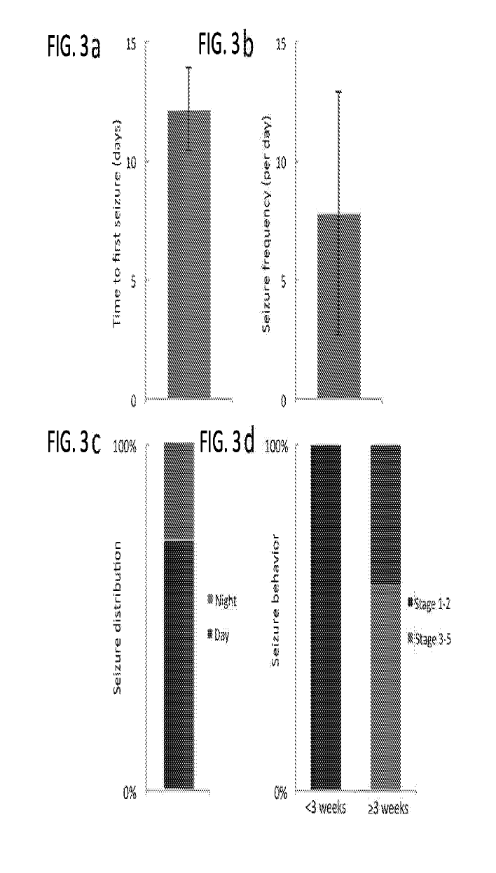

Continuous video-EEG monitoring revealed the first spontaneous seizures, which were nonconvulsive, to occur an average of 12.1 days after administration of 15 mg/kg KA and 0.75 mg/kg lorazepam (range 10-15 days) (FIG. 3A). Spontaneous seizures were detected in all animals, were typically 45-60 s long (FIG. 2A), and occurred at a frequency of 7.8 per animal per day during the first two weeks of spontaneous epilepsy (FIG. 3B). Seventy two percent of seizures occurred during the light phase (6:00 a.m. to 5:59 p.m.) (FIG. 3C). Intracerebral recordings obtained from the dentate gyrus demonstrated hippocampal involvement, for example, epileptiform discharging of granule cells (FIG. 2B). The corresponding, time-stamped video files revealed no overt seizure-like behavior, rather only freezing/staring. All spontaneous seizures that occurred during the first 2 weeks posttreatment were nonconvulsive, while starting at week 3, convulsive motor seizures were observed (FIG. 3D). Later spontaneous seizures (3 weeks post-treatment) also included behavioral manifestation, for example, mastication and forepaw clonus, corresponding to stages 3-5 on the Racine scale (Racine R J. Modification of seizure activity by electrical stimulation: II. Motor seizure. Electroencephalogr Clin Neurophysiol 1972; 32:281-294; herein incorporated by reference in its entirety). No spontaneous seizures were detected in any rats that received 15 mg/kg KA and 3 mg/kg lorazepam (4 weeks continuous video-EEG monitoring).

### Example 5

### Neuropathology Resembles (Refractory) Mesial TLE with Hippocampal Sclerosis

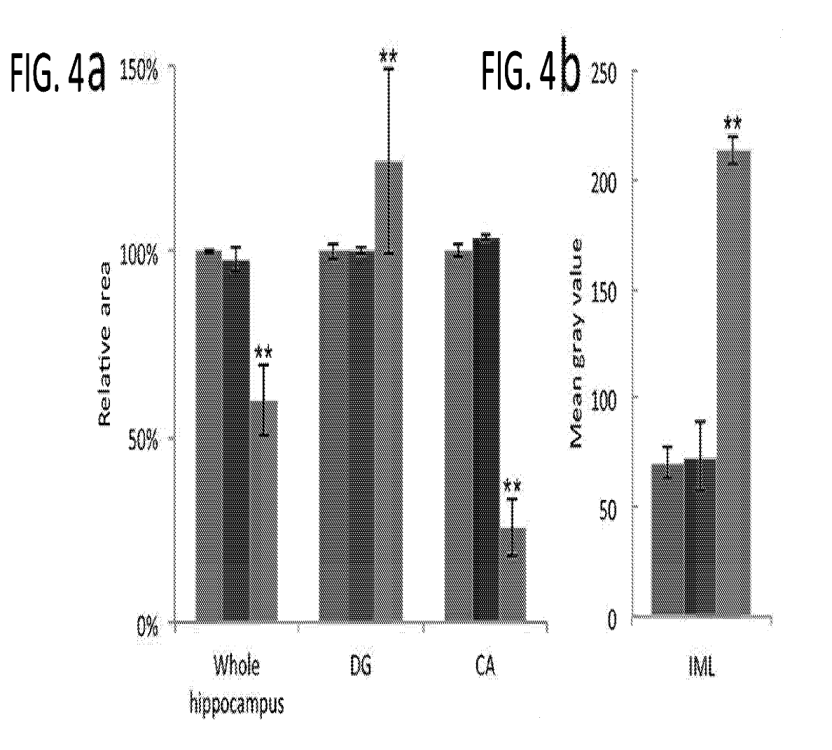

Hippocampal neuropathology of the non-human animal model of present invention closely mimics that seen in a subset of patients with mesial TLE whose seizures are refractory to drug treatment (International League Against Epilepsy [ILAE] Type I, (see for example Bluemcke I, Thom M, Aronica E, et al. International consensus classification of hippocampal sclerosis in temporal lobe epilepsy: a Task Force report front the ILAE Commission on Diagnostic Methods. Epilepsia 2013; 54:L315-1329; herein incorporated by reference in its entirety). In fact, ILAE Type I is the most common TLE pathology. Acutely, following administration of 15 mg/kg KA and 0.75 mg/kg lorazepam, pyramidal neurons in areas CA3 and CA1 were virtually wiped out, as were many neurons in the dentate hilus (FIG. 1B). Long-term histology 2 months) revealed hallmarks of mesial TLE, such as classic hippocampal sclerosis (FIG. 1D and FIG. 4A) and mossy fiber sprouting (FIG. 1F and FIG. 4B). Compared with control samples, atrophy was pronounced in the hippocampus overall (−40.0±9.6% mm2), specifically the hippocampus proper (−74.3±7.6% mm2), whereas the dentate gyrus was enlarged by at least 34% in three of four samples (all p-values <0.01). Although the thickness of the granule cell layer was consistently enlarged (124.7±25.0% of control), a phenomenon called “granule dispersion,” this expansion does not seem to drive the overall enlargement seen in the molecular layer (FIG. 1D).

Various modifications of the present invention, in addition to those shown and described herein, will be apparent to those skilled in the art of the above description. Such modifications are also intended to fall within the scope of the appended claims.

It is appreciated that all reagents are obtainable from commercial sources known in the art unless otherwise specified.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

The foregoing description is illustrative of particular aspects of the invention, but is not meant to be a limitation upon the practice thereof.

1. A non-human animal which has been administered a pharmaceutical combination comprising 10.0-30.3 mg of kainic acid per kg of the non-human animal and 0.25-1.4 mg of lorazepam per kg of the non-human animal, wherein the non-human animal exhibits a neurodegenerative disorder by said administration.

2. The non-human animal according to claim 1, wherein the neurodegenerative disorder comprises epilepsy, brain injury, spinal cord injury, bipolar disorder, trigeminal neuralgia, attention-deficit hyperactivity disorder, partial seizures, adjunctive therapy for partial, myoclonic, tonic-clonic seizures, schizophrenia, neuropathic pain, seizures, Tourette syndrome, Alzheimer's disease, autism, anxiety disorder, mania, phantom limb syndrome, complex regional pain syndrome, paroxysmal extreme pain disorder, neuromyotonia, intermittent explosive disorder, borderline personality disorder, myotonia congenita, Frontotemporal dementia, multiple sclerosis, Amyotrophic Lateral Sclerosis, Parkinson's disease, and Huntington's disease, traumatic brain injury, and post-traumatic stress disorder.

3. The non-human animal according to claim 1, wherein the neurodegenerative disorder is epilepsy.

4. The non-human animal according to claim 1, wherein the non-human animal does not exhibit status epilepticus and/or convulsive status epilepticus.

5. The non-human animal according to claim 1, wherein the non-human animal exhibits hippocampal sclerosis and spontaneous hippocampal-onset seizures.

6. The non-human animal according to claim 1, wherein the administered pharmaceutical combination comprises a first composition comprising 10.0-30.3 mg of kainic acid per kg of the non-human animal and a second composition comprising 0.25-1.4 mg of lorazepam per kg of the non-human animal.

7. The non-human animal according to claim 6, wherein the first composition comprising 10.0-30.3 mg of kainic acid per kg of the non-human is administered subcutaneously in a single dose, and the second composition comprising 0.25-1.4 mg of lorazepam per kg of the non-human animal is administered subcutaneously in a single dose.

8. The non-human animal according to claim 6, wherein the first composition comprising 10.0-30.3 mg of kainic acid per kg of the non-human and the second composition comprising 0.25-1.4 mg of lorazepam per kg of the non-human are administered concurrently.

9. The non-human animal according to claim 6, wherein the first composition comprising 10.0-30.3 mg of kainic acid per kg of the non-human and the second composition comprising 0.25-1.4 mg of lorazepam per kg of the non-human are administered together in a single dosage form.

10. The non-human animal according to claim 1, wherein the non-human animal is a rat.

11. A pharmaceutical combination comprising 10.0-30.3 mg of kainic acid per kg of a non-human animal and 0.25-1.4 mg of lorazepam per kg of the non-human animal.

12. (canceled)

13. (canceled)

14. (canceled)

15. (canceled)

16. The pharmaceutical combination according to claim 11, wherein the pharmaceutical combination comprises a first composition comprising 10.0-30.3 mg of kainic acid per kg of the non-human animal and a second composition comprising 0.25-1.4 mg of lorazepam per kg of the non-human animal.

17. The pharmaceutical formulation according to claim 16, wherein the first composition comprising 10.0-30.3 mg of kainic acid per kg of the non-human animal and the second composition comprising 0.25-1.4 mg of Lorazepam per kg of the non-human animal are formulated for subcutaneous injection.

18. A method of inducing a neurodegenerative disorder in a non-human animal, said method comprising:
administering a pharmaceutical combination comprising 10.0-30.3 mg of kainic acid per kg of the non-human animal and a 0.25-1.4 mg of lorazepam per kg of the non-human animal to the non-human animal;
inducing a neurodegenerative disorder in the non-human animal by said step of administering.

19. The method of inducing a neurodegenerative disorder in a non-human animal according to claim 18, wherein the neurodegenerative disorder comprises epilepsy, brain injury, spinal cord injury, bipolar disorder, trigeminal neuralgia, attention-deficit hyperactivity disorder, partial seizures, adjunctive therapy for partial, myoclonic, tonic-clonic seizures, schizophrenia, neuropathic pain, seizures, Tourette syndrome, Alzheimer's disease, autism, anxiety disorder, mania, phantom limb syndrome, complex regional pain syndrome, paroxysmal extreme pain disorder, neuromyotonia, intermittent explosive disorder, borderline personality disorder, myotonia congenita, Frontotemporal dementia, multiple sclerosis, Amyotrophic Lateral Sclerosis, Parkinson's disease, and Huntington's disease, traumatic brain injury, and post-traumatic stress disorder.

20. The method of inducing a neurodegenerative disorder in a non-human animal according to claim 18, wherein the neurodegenerative disorder is epilepsy.

21. The method of inducing a neurodegenerative disorder in a non-human animal according to claim 18, wherein the non-human animal does not exhibit status epilepticus and/or convulsive status epilepticus.

22. The method of inducing a neurodegenerative disorder in a non-human animal according to claim 18, wherein the non-human animal exhibits hippocampal sclerosis and spontaneous hippocampal-onset seizures.

23. The method of inducing a neurodegenerative disorder in a non-human animal according to claim 18, wherein the pharmaceutical combination comprises a first composition comprising 10.0-30.3 mg of Kainic acid per kg of the non-human animal and a second composition comprising 0.25-1.4 mg of Lorazepam per kg of the non-human animal.

24. The method of inducing a neurodegenerative disorder in a non-human animal according to claim 23, wherein the first composition comprising 10.0-30.3 mg of kainic acid per kg of the non-human is administered subcutaneously in a single dose, and the second composition comprising 0.25-1.4 mg of lorazepam per kg of the non-human animal is administered subcutaneously in a single dose.

25. The method of inducing a neurodegenerative disorder in a non-human animal according to claim 23, wherein the first composition comprising 10.0-30.3 mg of kainic acid per kg of the non-human and the second composition comprising 0.25-1.4 mg of lorazepam per kg of the non-human are administered concurrently.

26. The method of inducing a neurodegenerative disorder in a non-human animal according to claim 23, wherein the first composition comprising 10.0-30.3 mg of kainic acid per kg of the non-human and the second composition comprising 0.25-1.4 mg of lorazepam per kg of the non-human are administered together in a single dosage form.

27. The method of inducing a neurodegenerative disorder in a non-human animal according to claim 18, wherein the non-human animal is a rat.

28. A method of assaying the anti-neurodegenerative disorder efficacy of a compound or pharmaceutical composition, wherein said method comprises:
administering a compound or pharmaceutical composition postulated as having potential as an agent for treating a neurodegenerative disorder to a non-human animal which has been administered a pharmaceutical combination comprising 10.0-30.3 mg of kainic acid per kg of the non-human animal and 0.25-1.4 mg of lorazepam per kg of the non-human animal; and
determining the rate of occurrence and/or severity of neuron degeneration induced in said non-human animal, wherein a decreased rate of occurrence and/or severity of neuron degeneration is associated with anti-neurodegenerative efficacy of the compound or pharmaceutical composition.

29. The method of assaying the anti-neurodegenerative disorder efficacy of a compound or pharmaceutical composition according to claim 28, wherein the neurodegenerative disorder comprises epilepsy, brain injury, spinal cord injury, bipolar disorder, trigeminal neuralgia, attention-deficit hyperactivity disorder, partial seizures, adjunctive therapy for partial, myoclonic, tonic-clonic seizures, schizophrenia, neuropathic pain, seizures, Tourette syndrome, Alzheimer's disease, autism, anxiety disorder, mania, phantom limb syndrome, complex regional pain syndrome, paroxysmal extreme pain disorder, neuromyotonia, intermittent explosive disorder, borderline personality disorder, myotonia congenita, Frontotemporal dementia, multiple sclerosis, Amyotrophic Lateral Sclerosis, Parkinson's disease, and Huntington's disease, traumatic brain injury, and post-traumatic stress disorder.

30. The method of assaying the anti-neurodegenerative disorder efficacy of a compound or pharmaceutical composition according to claim 28, wherein the neurodegenerative disorder is epilepsy.

31. The method of assaying the anti-neurodegenerative disorder efficacy of a compound or pharmaceutical composition according to claim 28, wherein the non-human animal does not exhibit status epilepticus and/or convulsive status epilepticus.

32. The method of assaying the anti-neurodegenerative disorder efficacy of a compound or pharmaceutical composition according to claim 28, wherein the non-human animal exhibits hippocampal sclerosis and spontaneous hippocampal-onset seizures.

33. The method of assaying the anti-neurodegenerative disorder efficacy of a compound or pharmaceutical composition according to claim 28, wherein the administered pharmaceutical combination comprises a first composition comprising 10.0-30.3 mg of kainic acid per kg of the non-human animal and a second composition comprising 0.25-1.4 mg of lorazepam per kg of the non-human animal.

34. The method of assaying the anti-neurodegenerative disorder efficacy of a compound or pharmaceutical composition according to claim 33, wherein the first composition comprising 10.0-30.3 mg of kainic acid per kg of the non-human is administered subcutaneously in a single dose, and the second composition comprising 0.25-1.4 mg of lorazepam per kg of the non-human animal is administered subcutaneously in a single dose.

35. The method of assaying the anti-neurodegenerative disorder efficacy of a compound or pharmaceutical composition according to claims-33, wherein the first composition comprising 10.0-30.3 mg of kainic acid per kg of the non-human and the second composition comprising 0.25-1.4 mg of lorazepam per kg of the non-human are administered concurrently.

36. The method of assaying the anti-neurodegenerative disorder efficacy of a compound or pharmaceutical composition according to claim 33, wherein the first composition comprising 10.0-30.3 mg of kainic acid per kg of the non-human and the second composition comprising 0.25-1.4 mg of lorazepam per kg of the non-human are administered together in a single dosage form.

37. The method of assaying the anti-neurodegenerative disorder efficacy of a compound or pharmaceutical composition according to claim 28, wherein the non-human animal is a rat.

38. (canceled)
